(19) United States
(12) Patent Application Publication
(10) Pub. No.: US 2019/0227044 A1
(43) Pub. Date: Jul. 25, 2019

(54) ARTIFICIAL OLFACTORY SENSING SYSTEM AND MANUFACTURING METHOD OF THE SAME

(71) Applicants: Hitachi, Ltd., Tokyo (JP); The University of Tokyo, Tokyo (JP)

(72) Inventors: Masahiko ANDO, Tokyo (JP); Norifumi KAMESHIRO, Tokyo (JP); Tadashi OKUMURA, Tokyo (JP); Sanato NAGATA, Tokyo (JP); Ryohei KANZAKI, Tokyo (JP); Daigo TERUTSUKI, Tokyo (JP); Hidefumi MITSUNO, Tokyo (JP); Takeshi SAKURAI, Tokyo (JP)

(73) Assignees: HITACHI, LTD; The University of Tokyo

(21) Appl. No.: 16/250,793

(22) Filed: Jan. 17, 2019

(30) Foreign Application Priority Data
Jan. 19, 2018 (JP) 2018-007089

(51) Int. Cl. G01N 33/00

(52) U.S. Cl. CPC G01N 33/0032; G01N 33/0001

(57) ABSTRACT

An artificial olfactory sensing system includes a sensor unit. The sensor unit includes a semiconductor device equipped with a transistor and a sensor cell in which an olfactory receptor is manifested on a lipid film. A proton adsorption film is formed on a gate electrode of the transistor. A physiological aqueous solution is disposed on the proton adsorption film. Then, the sensor cell is disposed in the physiological aqueous solution. A proton is adsorbed onto the proton adsorption film. When the olfactory receptor recognizes an odor molecule, the positive ions in the physiological aqueous solution flow from an ion channel of the olfactory receptor into the sensor cell. As a result, the proton is dissociated from the proton adsorption film into the physiological aqueous solution, and the potential of the gate electrode is changed.

### BACKGROUND OF THE INVENTION

### 1. Field of the Invention

The invention relates to an artificial olfactory sensing system in which a biological material and a semiconductor material are assembled.

### 2. Description of the Related Art

Sensing techniques to artificially reproducing sensibility become essential techniques for protecting safety, health, and security in complex and diversified human societies and global environments. If an odor sensor (artificial olfactory sensing system) having high biological sensitivity is realized, odor information which has not been used up to now can be utilized, and may be applied to robots, automatic driving, medical treatment, risk prediction, disaster relief.

As an example of the artificial olfactory sensing system, WO 2017/122338 and “Odor-Sensitive Field Effect Transistor (OSFET) Based on Insect Cells Expressing Insect Odorant Receptors” disclose techniques in which a bio-technique and a semiconductor technique are combined. In the configuration of this technique, an electrical response generated when an olfactory receptor of an olfactory cell extracted from an organism recognizes an odor molecule is measured using an FET (Field-effect Transistor) (WO 2017/122338, and D. Terutsuki et al.: Odor-Sensitive Field Effect Transistor (OSFET) Based on Insect Cells Expressing Insect Odorant Receptors: Proc. MEMS 2017, pp. 394-397 (2017)).

### SUMMARY OF THE INVENTION

The inventor studies a technique of an artificial olfactory sensing system in which an electrical response according to the recognition of an odor molecule is detected with high sensitivity.

According to the configuration of the artificial olfactory sensing system and the manufacturing method thereof, the performance of the artificial olfactory sensing system is expected to be improved.

Other objects and new techniques will be cleared from the description of the specification and the accompanying drawings.

An artificial olfactory sensing system according to an embodiment includes a sensor unit. The sensor unit includes a transistor and a sensor cell in which an olfactory receptor is manifested on a lipid film. A first insulating film is formed on a gate electrode of the transistor, and an electrolytic aqueous solution is disposed on the first insulating film. Then, the sensor cell is disposed in the electrolytic aqueous solution, and a proton is adsorbed onto the first insulating film. When the olfactory receptor recognizes the odor molecule, positive ions in the electrolytic aqueous solution flow from an ion channel of the olfactory receptor into the sensor cell. As a result, the proton is dissociated from the first insulating film into the electrolytic aqueous solution, and the potential of the gate electrode is changed.

According to an embodiment, the performance of an artificial olfactory sensing system can be improved.

### BRIEF DESCRIPTION OF THE DRAWINGS

FIG. 1 is a diagram illustrating a configuration of an artificial olfactory sensing system of an embodiment;

FIG. 2 is a cross-sectional view illustrating main parts of a sensor unit of the artificial olfactory sensing system of an embodiment;

FIG. 3 is an enlarged cross-sectional view illustrating main parts of the sensor unit illustrated in FIG. 2;

FIG. 4 is a graph illustrating a voltage dependency of an extension gate electrode which is disposed in the sensor unit of the artificial olfactory sensing system of an embodiment;

FIG. 5 is a cross-sectional view illustrating main parts in a manufacturing process of a semiconductor device subsequent to FIG. 4;

FIG. 6 is a graph illustrating a temporal variation of a fluorescence where a green fluorescent protein in a sensor cell of the artificial olfactory sensing system of an embodiment is generated, and a graph illustrating a temporal variation of a potential of a gate electrode of the semiconductor device of the artificial olfactory sensing system;

FIG. 7 is a cross-sectional view illustrating main parts of the sensor unit of an artificial olfactory sensing system of a first investigation example;

FIG. 8 is a cross-sectional view illustrating main parts of the sensor unit of an artificial olfactory sensing system of a second investigation example;

FIG. 9 is an enlarged cross-sectional view illustrating main parts of the sensor unit of an artificial olfactory sensing system of a second embodiment; and

FIG. 10 is a diagram schematically illustrating molecules of a proton adsorption film of the second embodiment.

### DESCRIPTION OF THE PREFERRED EMBODIMENTS

Hereinafter, embodiments of the invention will be described in detail with reference to the drawings. Further, in the drawings for describing the embodiments, the members having the same function will be attached with the same symbol, and the redundant description thereof will be omitted. In addition, the descriptions on the same or similar portions will not be repeated in the following embodiments if not particularly necessary.

### First Embodiment

### <Configuration of Artificial Olfactory Sensing System>

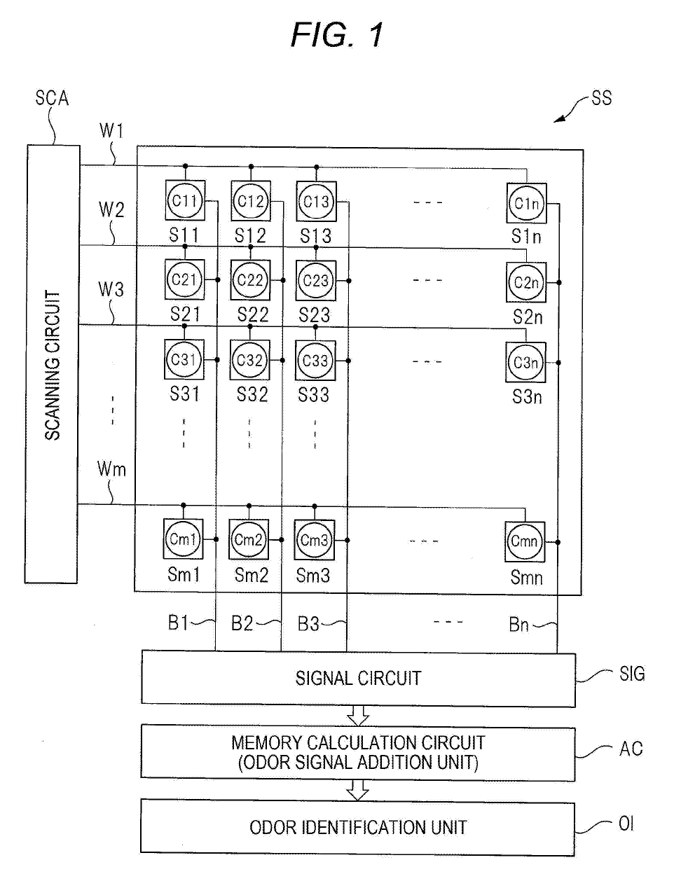

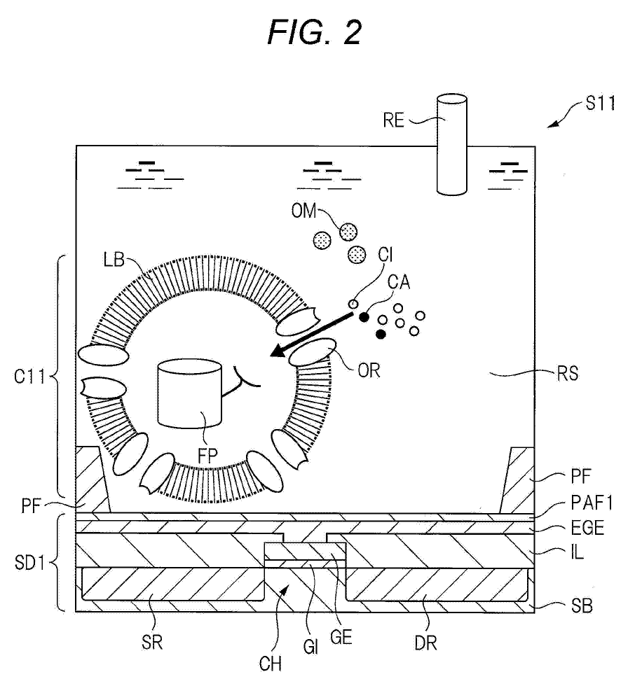

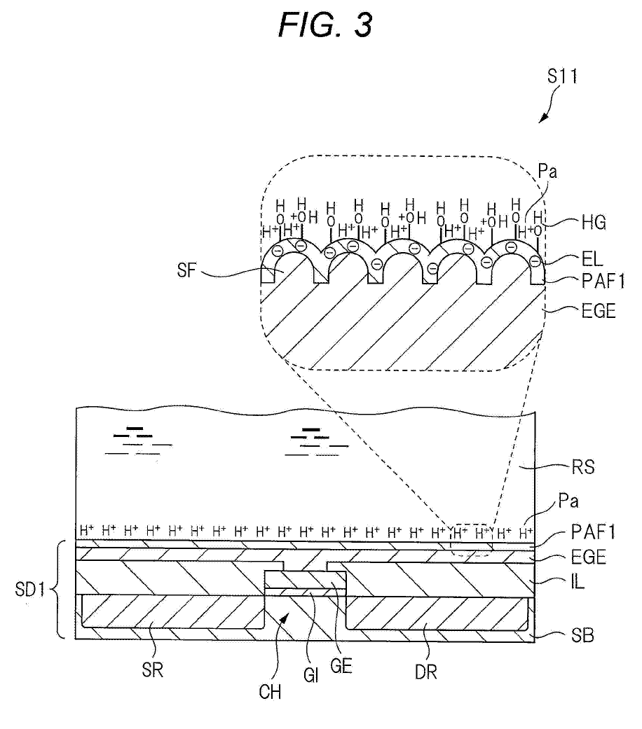

The configuration of an artificial olfactory sensing system in an embodiment will be described using FIGS. 1 to 3. FIG. 1 is a diagram illustrating a configuration of an artificial olfactory sensing system SS of this embodiment. FIG. 2 is a cross-sectional view illustrating main parts of a sensor unit S11 of the artificial olfactory sensing system of this embodiment. FIG. 3 is an enlarged cross-sectional view illustrating main parts of the sensor unit S11 illustrated in FIG. 2.

As illustrated in FIG. 1, m scanning lines Wi (i=1, . . . , m) and n signal lines Bj (j=1, . . . , n) are disposed to be crossed in the artificial olfactory sensing system SS of this embodiment. At the intersections between the m scanning lines Wi and the n signal lines Bj, sensor units Sij (i, j=1, 1, . . . , m, n) each are disposed in an m×n two-dimensional matrix shape. On each sensor unit Sij, each of sensor cells Cij (i, j=1, 1, . . . , m, n) is disposed. For example, in the case of m=1000 and n=1000, there are disposed 1,000,000 sensor units Sij in total. 1,000,000 sensor cells Cij each are disposed on the sensor units Sij. Further, as illustrated in FIGS. 1 and 2, the description will be given about a case where one sensor cell Cij is disposed on one sensor unit Sij, but the invention is not limited thereto. The plurality of sensor cells Cij may be disposed in one sensor unit Sij.

As illustrated in FIG. 1, the scanning line Wi is connected to a scanning circuit SCA, and the signal line Bj is connected to a signal circuit SIG. Then, the signal circuit SIG is connected to a memory calculation circuit (odor signal addition unit) AC, and the memory calculation circuit AC is connected to an odor identification unit OI.

FIG. 2 is a cross-sectional view illustrating main parts of the sensor unit S11 of the artificial olfactory sensing system SS illustrated in FIG. 1. FIG. 3 is an enlarged cross-sectional view illustrating main parts of the artificial olfactory sensing system illustrated in FIG. 2.

As illustrated in FIG. 2, the sensor unit S11 of this embodiment includes a semiconductor device SD1, a physiological aqueous solution (electrolytic aqueous solution) RS which is disposed on the semiconductor device SD1, and the sensor cell C11 which is disposed in the physiological aqueous solution RS.

As illustrated in FIGS. 2 and 3, the semiconductor device SD1 of this embodiment includes a substrate (semiconductor substrate) SB. The substrate SB is made of silicon (Si) for example. On the principal plane of the substrate SB, a MOSFET (Metal-Oxide-Semiconductor Field-effect Transistor) is formed as a semiconductor element. The MOSFET formed in the semiconductor device SD1 includes a source region SR and a drain region DR which are formed in the substrate SB, a channel region CH which is formed between the source region SR and the drain region DR, a gate insulating film GI which is formed on the channel region CH, and a gate electrode GE which is formed on the gate insulating film GI. The MOSFET may employ various types of sensor FETs. The semiconductor device SD1 is particularly called an ISFET (Ion Sensitive Field Effect Transistor).

An insulating layer IL is formed on the substrate SB to cover the MOSFET. The insulating layer IL is made of an oxide silicon film for example. Further, the insulating layer IL is not formed in the upper surface of a part of the gate electrode GE. An extension gate electrode (first conductor film) EGE is formed on the gate electrode GE and the insulating layer IL. The gate electrode GE and the extension gate electrode EGE are connected through the region where the insulating layer IL of the gate electrode is not formed. The extension gate electrode EGE occupies an area larger than at least the sensor cell C11 in top view. The extension gate electrode EGE is made of an aluminum (Al) film. The thickness of the extension gate electrode EGE is, for example, about 300 nm. Further, the gate electrode GE and the extension gate electrode EGE may be integrally formed.

In addition, a proton adsorption film (first insulating film) PAF1 is formed on the extension gate electrode EGE. The proton adsorption film PAF1 is formed in the same area as the extension gate electrode EGE in top view. The proton adsorption film PAF1 is made of an oxide aluminum (Al₂O₃) film. The thickness of the proton adsorption film PAF1 is, for example, about 5 nm or less. As illustrated in FIG. 3, the proton adsorption film PAF1 is porous. A surface SF of the proton adsorption film PAF1 has an uneven shape. Electrons EL are captured in the proton adsorption film PAF1, the surface SF of the proton adsorption film PAF1 is charged negatively. In other words, the proton adsorption film PAF1 has negatively fixed charges. The fixed charges are charges which do not move by an electric field but are fixed. In addition, a hydroxyl group (—OH) HG is bonded to an aluminum atom existing in the surface SF of the proton adsorption film PAF1. The surface SF of the proton adsorption film PAF1 is covered by the hydroxyl group HG. Then, a proton (hydrogen ion, H⁺) Pa is adsorbed to the surface SF of the proton adsorption film PAF1. Since the hydroxyl group HG exists in the surface SF of the proton adsorption film PAF1, the proton Pa is hydrogen-bonded to oxygen in the hydroxyl group HG so as to be stabilized. In addition, the proton adsorption film PAF1 comes into contact with the physiological aqueous solution RS. The physiological aqueous solution RS is an electrolytic aqueous solution containing Na⁺ and Ca²⁺. In addition, as illustrated in FIG. 2, a part of the proton adsorption film PAF1 (the end portion of the proton adsorption film PAF1 in top view) is covered by a protection film PF. The protection film PF is made of a silicon nitride film or a polyimide film for example. With the protection film PF, it is possible to suppress electrical crosstalk between the extension gate electrodes each of which is included in the adjacent sensor unit (for example, between the extension gate electrode EGE included in the sensor unit S11 and the extension gate electrode EGE included in the sensor unit S12). In particular, in a case where the thickness of the protection film PF is larger than an ion concentration change region in the surface of the extension gate electrode EGE (about Debye length to 1 μm described below), the electrical crosstalk can be effectively suppressed. Further, the description in FIG. 2 has been described about a case where the protection film PF is formed on the proton adsorption film PAF1, but the invention is not limited thereto. The protection film PF may be formed in the same layer as the proton adsorption film PAF1, that is, on the extension gate electrode EGE. In addition, the protection film PF may not be formed.

As illustrated in FIG. 2, the sensor cell C11 of this embodiment includes a lipid (double) film LB, an olfactory receptor OR which is manifested in the lipid film LB, and a calcium 2-valued ion (Ca²⁻) indication/green fluorescent protein FP (hereinafter, referred to as a green fluorescent protein FP) which is manifested in the sensor cell C11. The sensor cell C11 is soaked in the physiological aqueous solution RS. The sensor cell C11 comes into contact with the proton adsorption film PAF1 of the semiconductor device SD1. Alternatively, the sensor cell C11 floats in the physiological aqueous solution RS in a predetermined distance from the proton adsorption film PAF1 of the semiconductor device SD1. For example, a cell deprived from spodoptera frugiperda may be used as the sensor cell C11. A Sf21 cell or Sf9 cell is desirably employed. The Sf21 cell can stay alive on a wide temperature condition of 18 to 40° C. Since there is no need of using carbon dioxide to adjust pH of a culture solution, the Sf21 cell can be used semipermenantly. Therefore, the Sf21 cell is particularly desirable as the sensor cell C11. The sensor cell C11 is a spherical shape, and a diameter of the sensor cell C11 is about 20 μm.

Further, the configurations of the sensor units Sij other than the sensor unit S11 described above are similar to the configuration of the sensor unit S11, and the redundant description will be omitted. In other words, the configurations of the sensor cells Cij other than the sensor cell C11 are also similar to the configuration of the sensor cell C11.

### <Manufacturing Method of Artificial Olfactory Sensing System>

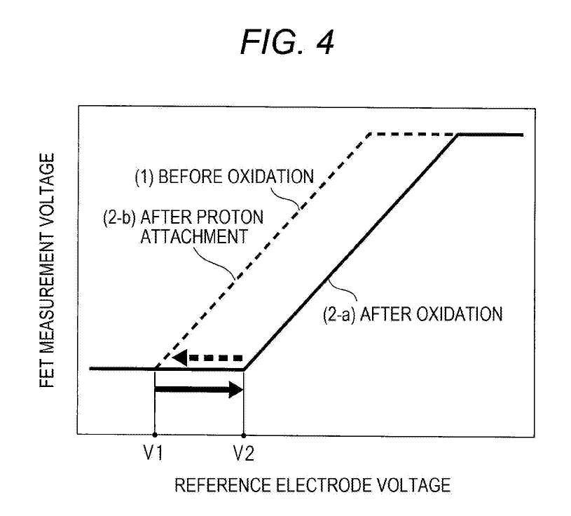

A manufacturing method of the artificial olfactory sensing system of this embodiment will be described in an order of processing. FIG. 4 is a graph illustrating a voltage dependency of the extension gate electrode which is disposed in the sensor unit of the artificial olfactory sensing system of this embodiment.

First, the substrate SB is prepared as illustrated in FIG. 2. For example, a silicon wafer is used as the substrate SB.

After a formation region of the MOSFET of the substrate SB (active region) is thermally oxidized to form a silicon oxide film, a polysilicon film is formed on the active region for example. Then, the polysilicon film and the silicon oxide film are patterned by a photolithography technique and a dry etching technique to form the gate electrode GE and the gate insulating film GI of the MOSFET. Further, p-type (or n-type) impurities (dopant) are ion-implanted to the substrate SB through self-alignment using the gate electrode GE as a mask. Thereafter, the impurities are diffused by thermal processing to form the source region SR and the drain region DR of the MOSFET in the substrate SB. Next, the insulating layer IL made of, for example, the silicon oxide film is formed on the substrate SB by a CVD (Chemical Vapor Deposition) method. Then, the insulating layer IL is patterned by the photolithography technique and the dry etching technique to expose the upper surface of a part of the gate electrode GE.

Through the above process, the MOSFET can be formed as a semiconductor element on the principal plane of the substrate SB. Further, the process herein may be substituted by preparing the substrate SB formed with the existing sensor FET.

Next, an aluminum film (not illustrated) is formed by a thickness of 300 nm on the gate electrode GE and the insulating layer IL by a PVD (Physical Vapor Deposition) method. Thereafter, the aluminum film is patterned in a square shape having one side of 32 μm in top view by a lift-off method.

Thereafter, oxygen (O₂) plasma processing is performed on the surface of the patterned aluminum film. The oxygen plasma processing is an application of a RIE (Reactive Ion Etching) method in which oxygen gas is applied with electromagnetic waves in a reaction chamber to generate plasma, and the aluminum film is simultaneously applied with radio-frequency voltage. Therefore, a self-bias potential is generated between the aluminum film and the plasma, and an ion species or a radical species in the plasma is accelerated to be brought into conflict with the sample. At that time, sputtering of oxygen-derived ions with respect to the aluminum film and an oxygen reaction of oxygen gas to the aluminum film occur at the same time.

With this configuration, the surface of the aluminum film is oxidized, and an aluminum oxide film is formed in the surface of the aluminum film. At this time, the portion (aluminum oxide film) oxidized by the oxygen plasma processing in the original aluminum film forms the proton adsorption film PAF1, and the portion not oxidized by the oxygen plasma processing forms the extension gate electrode EGE. As illustrated in FIG. 3, the proton adsorption film PAF1 is formed as a porous film by roughing the surface SF through the oxygen plasma processing. In addition, a number of chemical dangling bonds of the aluminum atoms are formed in the surface SF of the proton adsorption film PAF1. Then, a number of electrons are captured during the oxygen plasma processing, and the proton adsorption film PAF1 becomes a negative charged state after the oxygen plasma processing. In other words, the proton adsorption film PAF1 contains the negative fixed charges. The oxygen plasma processing of this embodiment is performed during 10 minutes on the condition that a flow rate of O₂ gas is 300 sccm (standard cubic centimeter per minute) and a radio-frequency bias power is set to 300 W.

Thereafter, the physiological aqueous solution RS containing Na⁺ and Ca²⁺ is disposed on the proton adsorption film PAF1, and the proton adsorption film PAF1 and the physiological aqueous solution RS come into contact with each other. With this configuration, the chemical dangling bond of the aluminum atoms existing in the surface SF of the proton adsorption film PAF1 reacts with water in the physiological aqueous solution RS. The hydroxyl group (—OH) HG is bonded to the chemical dangling bond of the aluminum atoms existing in the surface SF of the proton adsorption film PAF1. Then, since the proton adsorption film PAF1 is charged negatively, the proton (H⁺) Pa generated by the reaction between the chemical dangling bond and the water in the physiological aqueous solution RS is adsorbed to the proton adsorption film PAF1 by a Coulomb force. Thereafter, the proton Pa is stabilized by hydrogen-bonding to oxygen contained in the hydroxyl group HG. Therefore, the proton Pa enters a state of bonding the proton adsorption film PAF1 with priority higher than that of the other positive ions (Na⁺ and Ca²⁺) contained in the physiological aqueous solution RS.

Thereafter, the sensor cell C11 is introduced to the physiological aqueous solution RS which is disposed on the proton adsorption film PAF1. As described above, the sensor cell C11 does not necessarily come into contact with the proton adsorption film PAF1 of the semiconductor device SD1. In other words, the sensor cell may be in a state of floating in the physiological aqueous solution RS in a predetermined distance from the proton adsorption film PAF1 of the semiconductor device SD1. Further, the green fluorescent protein FP is manifested in the sensor cell C11 of this embodiment in advance using a genetic engineering method.

With the above process, the sensor unit S11 is completed. Further, the sensor units Sij other than the sensor unit S11 illustrated in FIG. 2 are formed through the similar process.

Thereafter, as illustrated in FIG. 1, the m scanning lines Wi and the n signal lines Bj are connected to the sensor units Sij which are disposed to be crossed. Thereafter, the scanning line Wi is connected to the scanning circuit SCA, and the signal line Bj is connected to the signal circuit SIG. Then, the signal circuit SIG is connected to the memory calculation circuit (odor signal addition unit) AC, and the memory calculation circuit AC is connected to the odor identification unit OI, so that the artificial olfactory sensing system SS of this embodiment is completed.

Herein, as described above, in order to confirm that the proton adsorption film PAF1 is negatively charged, a relation between a voltage (hereinafter, referred to as an FET measurement voltage) measured by the gate electrode GE of the MOSFET of the semiconductor device SD1 of this embodiment and a voltage to be applied to a reference electrode RE is investigated. Specifically, (1) in a state where the physiological aqueous solution RS is disposed on the aluminum film before the oxygen plasma processing is performed, and the physiological aqueous solution RS is brought to contact with the surface of the aluminum film before the oxygen plasma processing is performed, the reference electrode RE made of Ag/AgCl is inserted to the physiological aqueous solution RS (see FIG. 2), and the FET measurement voltage is measured when the voltage is applied to the reference electrode RE. In addition, (2) in a state where the physiological aqueous solution RS is disposed on the aluminum film after the oxygen plasma processing is performed, and the physiological aqueous solution RS is brought to contact with the surface of the aluminum film after the oxygen plasma processing is performed, that is, the proton adsorption film PAF1, the reference electrode RE made of Ag/AgCl is inserted to the physiological aqueous solution RS (see FIG. 2), and the FET measurement voltage is measured when the voltage is applied to the reference electrode RE. FIG. 4 illustrates the results.

As illustrated in FIG. 4, (1) when the voltage of the reference electrode RE becomes equal to or more than V1 in the aluminum film before the oxygen plasma processing is performed, the FET measurement voltage is monotonously increased. Herein, the voltage V1 becomes a threshold voltage in the aluminum film before the oxygen plasma processing. On the other hand, (2-a) when the voltage of the reference electrode RE becomes equal to or more than V2 in the aluminum film after the oxygen plasma processing, that is, the proton adsorption film PAF1, the FET measurement voltage is monotonously increased. Herein, the voltage V2 becomes the threshold voltage in the aluminum film after the oxygen plasma processing. A relation between the threshold voltage V1 and the threshold voltage V2 becomes V1<V2. In other words, the threshold voltage after the oxygen plasma processing is performed is shifted in the positive direction compared to the threshold voltage before the oxygen plasma processing is performed. From the result, as illustrated above, it can be confirmed that the proton adsorption film PAF1 is negatively charged.

Further, (2-a) is a state immediately after the physiological aqueous solution RS is brought to contact with the surface of the proton adsorption film PAF1. As time goes on from this state, the threshold voltage is gradually shifted from V2 in the negative direction, and finally equal to V1 (2-b). As described above, the chemical dangling bond existing in the surface SF of the proton adsorption film PAF1 reacts with the water in the physiological aqueous solution RS. The chemical dangling bond existing in the surface SF of the proton adsorption film PAF1 is bonded to the hydroxyl group (—OH) HG. Then, since the proton adsorption film PAF1 is charged negatively, the proton (H⁺) Pa generated by the reaction between the chemical dangling bond and the water in the physiological aqueous solution RS is adsorbed to the proton adsorption film PAF1 by a Coulomb force. Therefore, the protons as many as the holding charges are adsorbed to the proton adsorption film PAF1 immediately before the physiological aqueous solution RS is brought to contact, and the adsorption of the protons Pa is saturated. At this time, as the protons Pa come to be adsorbed to the proton adsorption film PAF1, the corresponding negative charges are canceled, and finally the charges are equalized. As a result, the threshold voltage of (2-b) is considered to be equal to the threshold voltage V1 of the aluminum film before (1) the oxygen plasma processing. In other words, the proton Pa attached to the proton adsorption film PAF1 can also be confirmed.

Further, the negatively charged proton adsorption film PAF1 formed by the oxygen plasma processing is confirmed also by a surface potential measurement in which an AFM (Atomic Force Microscope) is used. In addition, the surface SF of the proton adsorption film PAF1 covered with the hydroxyl group is confirmed by observing a vibration adsorption peak of the hydroxyl group using a Raman spectrometry equipment.

### <Operation Principle of Artificial Olfactory Sensing System>

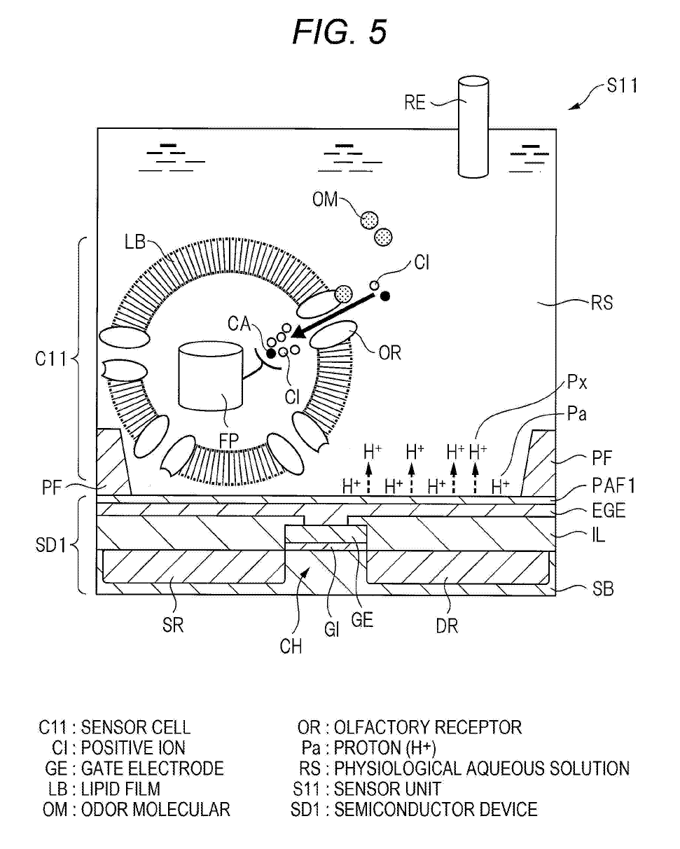

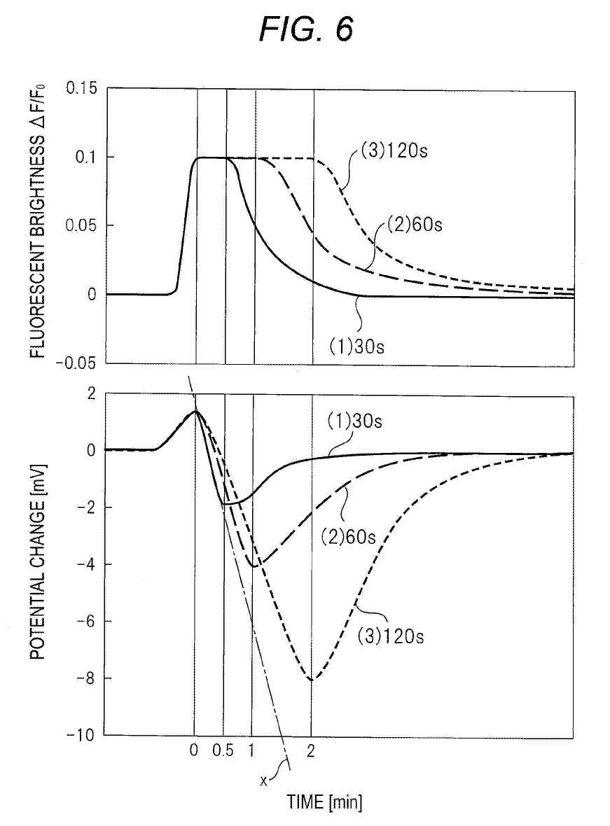

Hereinafter, an operation principle of the artificial olfactory sensing system of this embodiment will be described. FIG. 5 is a diagram illustrating an operation principle of the sensor unit S11 of the artificial olfactory sensing system of this embodiment. The upper diagram of FIG. 6 is a graph illustrating a temporal variation of the fluorescence where the green fluorescent protein FP in the sensor cell C11 of the artificial olfactory sensing system of this embodiment is generated. The lower diagram of FIG. 6 is a graph illustrating a temporal variation of the potential of the gate electrode GE of the semiconductor device SD1 of the artificial olfactory sensing system of this embodiment. Further, as described above, the configurations of the sensor units Sij other than the sensor unit S11 are similar to the configuration of the sensor unit S11, and the operation principle of the sensor unit Sij will be described by taking the sensor unit S11 as an example.

As illustrated in FIG. 5, the sensor unit S11 of this embodiment is in a state where the physiological aqueous solution RS is filled on the semiconductor device SD1. Then, the proton Pa is adsorbed to the surface SF of the proton adsorption film PAF1 of the semiconductor device SD1. In addition, the inside and the outside of the sensor cell C11 existing in the physiological aqueous solution RS are separated by the lipid film LB. With the operation of an ion pump (not illustrated) manifested in the surface of the lipid film LB, the concentration of the positive ions (H^{|}, Na^{|}, Ca^{2|}, etc.) in the cell is kept lower than the outside of the cell.

In the above state, it will be considered a case where an odor molecule OM is introduced into the physiological aqueous solution RS. When the olfactory receptor OR manifested in the lipid film LB captures and recognizes the odor molecule OM, an ion channel of the olfactory receptor OR is opened, and the positive ions including Ca²⁺ in the physiological aqueous solution RS flow into the sensor cell C11.

In the sensor cell C11 of this embodiment, the green fluorescent protein FP is manifested. Therefore, when the olfactory receptor OR captures and recognizes the odor molecule OM, and the positive ions including Ca²⁺ flow into the sensor cell C11, Ca²⁺ is captured in the green fluorescent protein FP, and the green fluorescence is increased.

Herein, the upper diagram of FIG. 6 illustrates a change in fluorescent brightness of the green fluorescent protein FP in a case where a stimulus (continuing) time of the odor molecule OM is set to (1) 30 s, (2) 60 s, and (3) 120 s. As illustrated in the upper diagram of FIG. 6, the fluorescence of the green fluorescent protein FP according to the recognization of the odor molecule OM is steeply increased as the stimulus of the odor molecule OM starts regardless of the stimulus (continuing) time of the odor molecule OM. Then, the fluorescence of the green fluorescent protein FP according to the recognition of the odor molecule OM is set to a constant value in the stimulus (continuing) time of the odor molecule OM. Then, the fluorescence of the green fluorescent protein FP according to the recognition of the odor molecule OM is gradually decreased as the stimulus of the odor molecule OM ends. With this configuration, in a case where the stimulus (continuing) time of the odor molecule is not recognized, the stimulus (continuing) time of the odor molecule can be grasped by measuring a time when the fluorescence of the green fluorescent protein FP is a constant value. In addition, a timing of starting the stimulus of the odor molecule and a timing of completing the stimulus of the odor molecule can also be grasped.

On the other hand, as illustrated in FIG. 5, the positive ions in the physiological aqueous solution RS flow into the sensor cell C11, so that the concentration of the positive ions in the physiological aqueous solution RS is lowered. In order to compensate the positive ions in the physiological aqueous solution RS, the proton Pa adsorbed to the surface SF of the proton adsorption film PAF1 of the semiconductor device SD1 is dissociated from the proton adsorption film PAF1, and emitted into the physiological aqueous solution RS (in FIG. 5, the dissociated proton is denoted by Px). As a result, the potential in the sensor cell C11 is shifted in the positive direction, and the potential of the surface SF of the proton adsorption film PAF1 is shifted in the negative direction.

With this configuration, the potentials of the extension gate electrode EGE and the gate electrode GE integrally formed with the proton adsorption film PAF1 are shifted in the negative direction. In a case where the MOSFET of the semiconductor device SD1 is a p-channel MOSFET, the carrier charges are accumulated in the channel region CH, and the current comes to flow between the source region SR and the drain region DR (ON state).

Herein, the change in potential of the gate electrode GE in a case where the stimulus (continuing) time caused by the odor molecule OM is set to (1) 30 s, (2) 60 s, and (3) 120 s is illustrated in the lower diagram of FIG. 6. Comparing the change of the fluorescence of the green fluorescent protein FP of the upper diagram of FIG. 6 and the change in potential of the gate electrode GE of the lower diagram of FIG. 6, the change in potential of the gate electrode GE illustrated in the lower diagram of FIG. 6 and the stimulus (continuing) time caused by the odor molecule OM can correspond. First, as described above, from the measurement result of the fluorescent brightness of the green fluorescent protein FP, the stimulus (continuing) time of the odor molecule, the timing of starting the stimulus of the odor molecule, and the timing of completing the stimulus of the odor molecule can be grasped. On the basis of the result, a change with time of the potential of the gate electrode GE is analyzed. As a result, as illustrated in the lower diagram of FIG. 6, the stimulus of the odor molecule OM starts and the potential of the gate electrode GE is monotonously reduced (monotonously increase in the negative direction). The potential is gradually increased in the positive direction as the stimulus of the odor molecule OM is completed, and finally returns to the original value. Therefore, it is known that a time during which the potential of the gate electrode GE is monotonously reduced is the stimulus (continuing) time of the odor molecule. Therefore, without measuring the fluorescent brightness of the green fluorescent protein FP, the stimulus (continuing) time of the odor molecule can be measured from the change in potential of the gate electrode GE.

Subsequently, the description will be given about a process after the change in potential of the gate electrode GE is observed in the artificial olfactory sensing system of this embodiment.

In the artificial olfactory sensing system of this embodiment, a sensor cell which responds to different types of odor molecules is prepared, and disposed in the sensor unit in which the same types of the sensor cells are connected to the same scanning line. For example, the sensor cells responding to an odor molecule OM1 are C11, C12, and C13, the sensor cells responding to an odor molecule OM2 are C21, C22, and C23, and the sensor cells responding to an odor molecule OM3 are C31, C32, and C33. Then, the sensor cells C11, C12, and C13 are connected to the scanning line W1. The sensor cells C21, C22, and C23 are connected to the scanning line W2. The sensor cells C31, C32, and C33 are connected to the scanning line W3.

In this case, the sensor cells C11, C12, and C13 in the sensor units S11, S12, and S13 respond to a certain type of the odor molecule OM1. The MOSFETs of the sensor units S11, S12, and S13 connected to the same scanning line W1 are simultaneously turned on. Therefore, an output signal (or current pulse width) caused by the sensor cells disposed on the same scanning line is received by the signal circuit SIG, and the addition is performed by the memory calculation circuit AC. Therefore, the output signals of the same types of the sensor cells can be added in the scanning period. The type of the odor molecule can be specified, and the concentration of the odor molecule can be measured in the odor identification unit OI on the basis of the output signal added in the memory calculation circuit AC.

### <Circumstances of Investigation>

### [First Investigation Example]

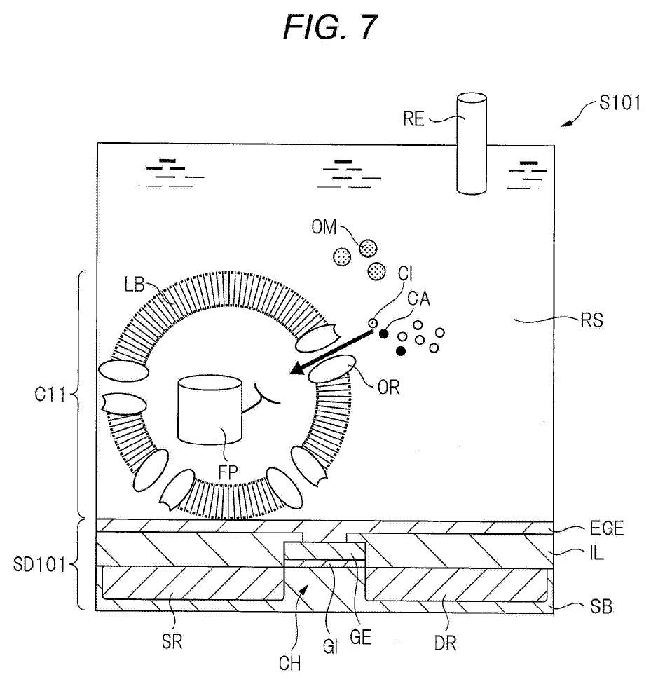

The configuration of the artificial olfactory sensing system of a first investigation example studied by the inventor will be described using FIG. 7. FIG. 7 is a cross-sectional view illustrating main parts of a sensor unit S101 of the artificial olfactory sensing system of the first investigation example.

In the artificial olfactory sensing system of the first investigation example, the configuration of the sensor unit which is one of the components is different from the configuration of the sensor unit of the artificial olfactory sensing system of this embodiment. The other configurations of the artificial olfactory sensing system of the first investigation example are the same as those of the artificial olfactory sensing system of this embodiment, and the redundant description will be omitted.

As illustrated in FIG. 7, the sensor unit S101 of the first investigation example includes a semiconductor device SD101, and the sensor cell C11 which is disposed on the semiconductor device SD101. In the semiconductor device SD101 of the first investigation example, the proton adsorption film is not formed on the extension gate electrode EGE. Then, the sensor cell C11 abuts on the extension gate electrode EGE. This configuration is different from the first investigation example and this embodiment.

Further, in the sensor unit of the artificial olfactory sensing system of the first investigation example, the configuration of the sensor unit other than the sensor unit S101 described above are similar to the configuration of the sensor unit S101, and the redundant description will be omitted.

In addition, in the manufacturing method of the artificial olfactory sensing system of the first investigation example, the aluminum film (not illustrated) is patterned on the gate electrode GE and the insulating layer IL similar to this embodiment. However, in the first investigation example, the surface of the patterned aluminum film is not subjected to an oxygen (O₂) plasma processing. In other words, the aluminum film itself serves as the extension gate electrode EGE. The above configuration of the first investigation example is different from this embodiment.

Next, the operation principle of the artificial olfactory sensing system of the first investigation example will be described.

Similar to this embodiment, the sensor unit S101 of the first investigation example is in a state where the physiological aqueous solution RS is filled on the semiconductor device SD101. In addition, the inside and the outside of the sensor cell C11 existing in the physiological aqueous solution RS are separated by the lipid film LB. With the operation of an ion pump (not illustrated) manifested in the surface of the lipid film LB, the concentration of the positive ions in the cell is kept lower than the outside of the cell. On the other hand, in the first investigation example, the sensor cell C11 abuts on the extension gate electrode EGE of the semiconductor device SD101.

In the above state, it will be considered a case where an odor molecule OM is introduced into the physiological aqueous solution RS. When the olfactory receptor OR manifested in the lipid film LB captures and recognizes the odor molecule OM, an ion channel of the olfactory receptor OR is opened, and the positive ions including Ca²⁺ in the physiological aqueous solution RS flow into the sensor cell C11.

Herein, the positive ions in the physiological aqueous solution RS flow into the sensor cell C11, and the potential in the sensor cell C11 is shifted in the positive direction. The change in potential is transferred to the extension gate electrode EGE through the lipid film LB, and the potential of the extension gate electrode EGE is shifted in the positive direction.

With this configuration, the potentials of the extension gate electrode EGE and the gate electrode GE are shifted in the positive direction. In a case where the MOSFET of the semiconductor device SD101 is an n-channel MOSFET, the carrier charges are accumulated in the channel region CH, and the current comes to flow between the source region SR and the drain region DR (ON state).

The processes after the change in potential of the gate electrode GE in the first investigation example is observed are similar to those of this embodiment, and the redundant description will be omitted.

Hereinafter, problems on the artificial olfactory sensing system of the first investigation example found out by the inventor will be described.

As described above, the sensor cell C11 is formed in a spherical shape, and it is difficult to perform contacting and covering with respect to the whole surface of the flat extension gate electrode EGE. Therefore, the potential shifting of the sensor cell C11 caused by the positive ions in the physiological aqueous solution RS flowing into the sensor cell C11 is not possible to be quantitatively detected by the extension gate electrode EGE.

In addition, it is also considered that the sensor cell C11 does not come into contact with the extension gate electrode EGE, and a gap is made between the sensor cell C11 and the extension gate electrode EGE. In this case, the physiological aqueous solution RS is interposed between the sensor cell C11 and the extension gate electrode EGE, and the potential shifting of the sensor cell C11 is not transferred to the extension gate electrode EGE.

As described above, in the artificial olfactory sensing system of the first investigation example, the influence on the detection sensitivity of the odor molecule caused by the surrounding environment of the sensor cell C11 is large, and the odor molecule is not possible to be stably detected.

### [Second Investigation Example]

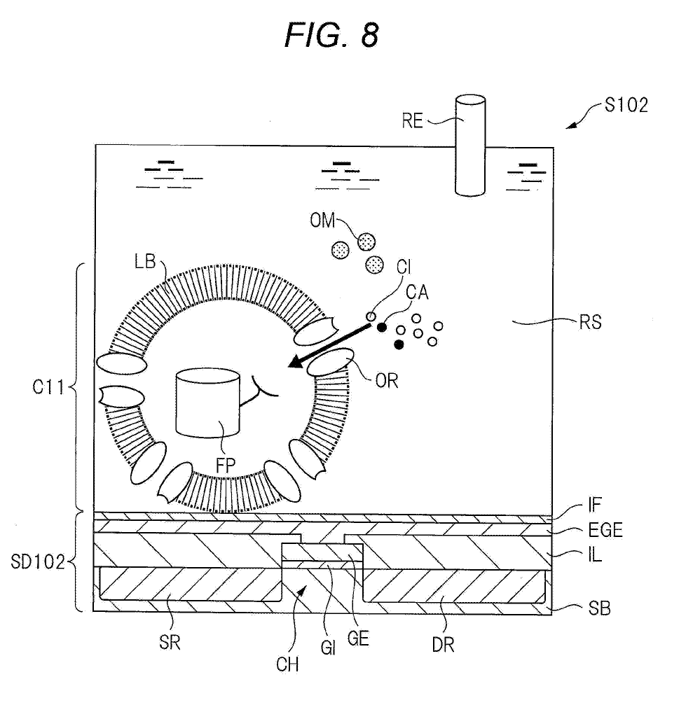

The configuration of the artificial olfactory sensing system of a second investigation example studied by the inventor will be described using FIG. 8. FIG. 8 is a cross-sectional view illustrating main parts of a sensor unit S102 of the artificial olfactory sensing system of the second investigation example.

In the artificial olfactory sensing system of the second investigation example, the configuration of the sensor unit which is one of the components is different from the configuration of the sensor unit of the artificial olfactory sensing system of this embodiment. The other configurations of the artificial olfactory sensing system of the second investigation example are the same as those of the artificial olfactory sensing system of this embodiment, and the redundant description will be omitted.

As illustrated in FIG. 8, the sensor unit S102 of the second investigation example includes a semiconductor device SD102, and the sensor cell C11 which is disposed on the semiconductor device SD102. In the semiconductor device SD102 of the second investigation example, an insulating film IF is formed on the extension gate electrode EGE instead of the proton adsorption film. The insulating film IF is made of the aluminum oxide film, but not porous. The surface of the insulating film is smooth compared to the proton adsorption film PAF1 of this embodiment. In addition, the insulating film IF does not capture electrons, and the surface thereof is not charged. Then, the surface of the insulating film IF is not covered by the hydroxyl group, and the proton is not adsorbed. In addition, the sensor cell C11 abuts on the insulating film IF. These configurations of the second investigation example are different from this embodiment.

Further, in the sensor units of the artificial olfactory sensing system of the second investigation example, the configurations of the sensor units other than the sensor unit S102 described above are similar to that of the sensor unit S102, and the redundant description will be omitted.

In addition, in the manufacturing method of the artificial olfactory sensing system of the second investigation example, the aluminum film (not illustrated) is formed on the gate electrode GE and the insulating layer IL, and patterned similar to this embodiment. Herein, in the second investigation example, for example, the surface of the patterned aluminum film is oxidized by a thermal oxidation method, and the aluminum oxide film is formed in the surface of the aluminum film. At this time, the portion (aluminum oxide film) among the original aluminum film oxidized by the thermal oxidation method forms the insulating film IF. The portion not oxidized by the thermal oxidation method forms the extension gate electrode EGE.

Further, for example, the surface of the insulating film IF is mostly not roughened by the thermal oxidation method. Therefore, the surface of the insulating film IF is smooth compared to the surface of the proton adsorption film PAF1 of this embodiment. In addition, the insulating film IF is not negatively charged. In addition, the chemical dangling bond of the aluminum atoms is mostly not formed in the surface of the insulating film IF. As a result, the physiological aqueous solution RS is disposed on the insulating film IF, and even if the insulating film IF and the physiological aqueous solution RS come into contact, the surface of the insulating film IF is not covered by the hydroxyl group, and the proton is also not absorbed to the surface of the insulating film IF.

Further, the aluminum oxide film is formed directly on the aluminum film by a sputtering method targeting the aluminum oxide, and the aluminum oxide film may be used as the insulating film IF. Even in this case, the formed aluminum oxide film is formed as a highly dense film. Therefore, the surface of the insulating film IF is smooth compared to the surface of the proton adsorption film PAF1 of this embodiment. In addition, the insulating film IF is not negatively charged. Then, the chemical dangling bond of the aluminum atoms is mostly not formed in the surface of the insulating film IF. As a result, even in this case, the physiological aqueous solution RS is disposed on the insulating film IF, and even if the insulating film IF and the physiological aqueous solution RS come into contact, the surface of the insulating film IF is not covered by the hydroxyl group, and the protons are also not adsorbed to the surface of the insulating film IF. The above configurations of the second investigation example are different from this embodiment.

Next, an operation principle of the artificial olfactory sensing system of the second investigation example will be described.

Similar to this embodiment, the sensor unit S102 of the second investigation example is in a state where the physiological aqueous solution RS is filled on the semiconductor device SD102. In addition, the inside and the outside of the sensor cell C11 existing in the physiological aqueous solution RS are separated by the lipid film LB. With the operation of an ion pump (not illustrated) manifested in the surface of the lipid film LB, the concentration of the positive ions in the cell is kept lower than the outside of the cell. On the other hand, in the second investigation example, the sensor cell C11 comes to contact with the insulating film IF of the semiconductor device SD102.

In the above state, it will be considered a case where an odor molecule OM is introduced into the physiological aqueous solution RS. When the olfactory receptor OR manifested in the lipid film LB captures and recognizes the odor molecule OM, an ion channel of the olfactory receptor OR is opened, and the positive ions including Ca²⁺ in the physiological aqueous solution RS flow into the sensor cell C11.

Herein, the positive ions in the physiological aqueous solution RS flow into the sensor cell C11, and the potential in the sensor cell C11 is shifted in the positive direction. The change in potential is transferred to the extension gate electrode EGE through the lipid film LB and the insulating film IF, and the potential of the extension gate electrode EGE is shifted in the positive direction.

With this configuration, the potentials of the extension gate electrode EGE and the gate electrode GE are shifted in the positive direction. In a case where the MOSFET of the semiconductor device SD102 is an n-channel MOSFET, the carrier charges are accumulated in the channel region CH, and the current flows between the source region SR and the drain region DR (ON state).

The processes after the change in potential of the gate electrode GE in the second investigation example is observed are similar to those of this embodiment, and the redundant description will be omitted.

Hereinafter, the problems found out in the artificial olfactory sensing system of the second investigation example by the inventor will be described.

As described above, the sensor cell C11 is formed in a spherical shape, and it is difficult to perform contacting and covering with respect to the whole surface of the flat extension gate electrode EGE. Therefore, in the first investigation example, the potential shift of the sensor cell C11 caused by the positive ions in the physiological aqueous solution RS flowing into the sensor cell C11 is not effectively transferred to the extension gate electrode EGE.

Herein, in the semiconductor device SD102 of the second investigation example, the insulating film IF is formed on the extension gate electrode EGE. Therefore, the insulating film IF is interposed between the sensor cell C11 and the extension gate electrode EGE. With this configuration, it can be seen that a weak potential response signal of the sensor cell C11 can be amplified.

On the other hand, as described above, it is also considered that the sensor cell C11 does not come into contact with the insulating film IF, and a gap is generated between the sensor cell C11 and the insulating film IF. In this case, the physiological aqueous solution RS is interposed between the sensor cell C11 and the insulating film IF, and the weak potential response signal of the sensor cell C11 caused by the insulating film IF is not effectively amplified.

As described above, in the artificial olfactory sensing system of the second investigation example, the distance between the sensor cell C11 and the insulating film IF is largely affected with respect to the detection sensitivity of the odor molecule, and the odor molecule is not possible to be stably detected. Therefore, the configuration and the manufacturing method of the artificial olfactory sensing system have been studied to effectively and stably transfer the change in potential of the sensor cell to the semiconductor device. It is desirable that the performance of the artificial olfactory sensing system is improved.

### <Primary Characteristics and Effects of Embodiment>

Hereinafter, the primary characteristics and effects of this embodiment will be described. One of the primary characteristics of this embodiment is that the proton adsorption film PAF1 made of the aluminum oxide film is formed on the extension gate electrode EGE of the semiconductor device SD1 as illustrated in FIG. 2. Then, as illustrated in FIG. 3, the proton adsorption film PAF1 is porous. The surface SF of the proton adsorption film PAF1 includes an uneven shape. In addition, in the proton adsorption film PAF1, the electron EL is captured, and the surface SF of the proton adsorption film PAF1 is negatively charged. In addition, the surface SF of the proton adsorption film PAF1 is covered by the hydroxyl group HG. Then, the proton Pa is adsorbed to the surface SF of the proton adsorption film PAF1 through the hydroxyl group HG.

In addition, in the manufacturing method of the semiconductor device SD1 of this embodiment, the oxygen (O₂) plasma processing is performed on the surface of the aluminum film, the porous aluminum oxide film is formed in the surface of the aluminum film, and the aluminum oxide film is used as the proton adsorption film PAF1. A number of chemical dangling bonds of the aluminum atoms are formed in the surface SF of the proton adsorption film PAF1. Then, a number of electrons are captured during the oxygen plasma processing, and the proton adsorption film PAF1 becomes a negative charged state after the oxygen plasma processing. Thereafter, the proton adsorption film PAF1 and the physiological aqueous solution RS are brought to contact. With this configuration, the chemical dangling bond of the aluminum atoms existing in the surface SF of the proton adsorption film PAF1 reacts with the water in the physiological aqueous solution RS. The hydroxyl group HG is bonded to the chemical dangling bond of the aluminum atoms existing in the surface SF of the proton adsorption film PAF1. Then, since the proton adsorption film PAF1 is negatively charged, the proton Pa generated in the reaction between the chemical dangling bond and the water in the physiological aqueous solution RS is adsorbed to the proton adsorption film PAF1.

In this embodiment, with such a configuration and manufacturing process, the performance of the artificial olfactory sensing system can be improved. Hereinafter, the reasons will be specifically described.

The sensor unit S11 of the artificial olfactory sensing system of this embodiment is in a state where, as illustrated in FIG. 5, the proton Pa is adsorbed to the surface SF of the proton adsorption film PAF1 of the semiconductor device SD1. Therefore, as illustrated in FIG. 5, in a case where the positive ions in the physiological aqueous solution RS flow into the sensor cell C11, and the concentration of the positive ions in the physiological aqueous solution RS is lowered, the proton Pa adsorbed to the surface SF of the proton adsorption film PAF1 of the semiconductor device SD1 is dissociated from the proton adsorption film PAF1, and emitted into the physiological aqueous solution RS in order to compensate the positive ions in the physiological aqueous solution RS. As a result, the potential in the sensor cell C11 is shifted in the positive direction, and the potential of the surface SF of the proton adsorption film PAF1 is shifted in the negative direction. With this configuration, the potentials of the extension gate electrode EGE and the gate electrode GE integrally formed with the proton adsorption film PAF1 are shifted in the negative direction.

In this way, in this embodiment, the potential change of the sensor cell C11 itself is not measured as described in the first and second investigation examples in electrical response to the odor molecule, but the change in potential of the gate electrode GE is measured according to the change in amount of the protons Pa existing on the proton adsorption film PAF1. The distance between the extension gate electrode EGE connected to the gate electrode and the proton adsorption film PAF1 is constant. The distance is about 5 nm and extremely small. Therefore, the change in potential of the gate electrode GE according to the change in amount of the protons Pa existing on the proton adsorption film PAF1 shows a stable change with high sensitivity and reproducibility.

In addition, in this embodiment, the potential change of the sensor cell C11 itself is not measured. Therefore, the sensor cell C11 does not necessarily come into contact with the proton adsorption film PAF1, and the influence of the distance between the sensor cell C11 and the proton adsorption film PAF1 with respect to the change in potential of the gate electrode GE is also small. Therefore, in this embodiment, the electrical response to the odor molecule can be observed with high sensitivity and reproducibility, and the performance of the artificial olfactory sensing system can be improved.

In addition, in this embodiment, it is considered that a ratio of the change to the negative direction of the potential of the gate electrode GE according to the stimulus of the odor molecule is limited to the dissociation speed of the proton Pa from the proton adsorption film PAF1. In addition, as the concentration of the positive ions in the physiological aqueous solution RS existing on the proton adsorption film PAF1 is lowered, the dissociation speed of the proton Pa from the proton adsorption film PAF1 becomes large. Then, as the inflow to the sensor cell C11 is large, the concentration of the positive ions in the physiological aqueous solution RS is lowered. In addition, as the concentration of the odor molecule is high, the inflow to the sensor cell C11 becomes large. From the above description, it can be seen that the ratio of the change in potential of the gate electrode GE according to the stimulus of the odor molecule becomes large in proportion to the concentration of the odor molecule. Therefore, if a relation between the concentration of the odor molecule and the ratio (that is, a time differential of the potential change) of the change to the negative direction of the potential of the gate electrode GE according to the stimulus of the odor molecule is obtained using the odor molecule of which the concentration is known already, the concentration of the odor molecule can be measured from the ratio of the change to the negative direction of the potential of the gate electrode GE according to the stimulus of the odor molecule.

Specifically, as illustrated in the lower diagram of FIG. 6, the potential of the gate electrode GE is monotonously decreased (monotonously increased in the negative direction) as the stimulus of the odor molecule starts. Therefore, for example, in a case where the concentration of the odor molecule is obtained when the stimulus (continuing) time of the odor molecule is (1) 30 s, a straight line x approximating to the monotonously decreasing portion of the graph of (1) 30 s is drawn, and the slope of the straight line x is obtained. Then, a relation between the slope of the straight line x and the concentration of the odor molecule is set in advance with reference to a calibration line for example, so that the concentration of the odor molecule can be measured.

In addition, in the manufacturing method of the artificial olfactory sensing system of this embodiment, the oxygen plasma processing is performed on the surface of the aluminum film, the porous aluminum oxide film is formed in the surface of the aluminum film, and the aluminum oxide film is used as the proton adsorption film PAF1.

It can be seen that, when the film density of the aluminum oxide film is lowered, the holding negative fixed charges become large. In this embodiment, the oxygen plasma processing is employed as the formation method of the aluminum oxide film. In the oxygen plasma processing, the sputtering of oxygen-derived ions with respect to the aluminum film occurs, so that it is possible to form the porous aluminum oxide film of which the surface is rough. Further, the electrons generated by the oxygen plasma processing are captured by the aluminum oxide film. With such a configuration, the aluminum oxide film formed by the oxygen plasma processing contains many negative fixed charges compared to the aluminum oxide film which is formed by the thermal oxidation method for example. As a result, the proton adsorption film PAF1 of this embodiment can make the proton (H⁺) Pa adsorbed by the negative fixed charges therein.

In addition, in the oxygen plasma processing, a number of chemical dangling bonds of the aluminum atoms are formed in the surface of the aluminum film by the sputtering caused by oxygen-derived ions. Therefore, since the aluminum film after the oxygen plasma processing (that is, the proton adsorption film PAF1) is brought to contact with the physiological aqueous solution RS, the chemical dangling bond of the aluminum atoms reacts with the water in the physiological aqueous solution RS, and the hydroxyl group HG is bonded to the chemical dangling bond of the aluminum atoms. Since the surface SF of the proton adsorption film PAF1 is covered with the hydroxyl group HG, the proton Pa adsorbed to the proton adsorption film PAF1 is hydrogen-bonded to the hydroxyl group HG so as to be stabilized. The surface SF of the proton adsorption film PAF1 can be kept in a state where the proton Pa is covered.

Further, as described above, in this embodiment, the sensor cell C11 may float in the physiological aqueous solution RS in a predetermined distance from the proton adsorption film PAF1 of the semiconductor device SD1. However, in order for the proton Pa adsorbed to the surface of the proton adsorption film PAF1 to be influenced by the positive ions flowing into the sensor cell C11, the proton adsorption film PAF1 necessarily exists within the thickness (Debye length) of an electrical double layer which is formed by the positive ions flowing to the sensor cell C11. In general, in a case where the concentration of the positive ions is about 10⁻¹ to 10⁻⁵ M (mol/L), the Debye length is about 1 to 100 nm. Therefore, the distance between the sensor cell C11 and the proton adsorption film PAF1 is desirably about 100 nm or less.

### Second Embodiment

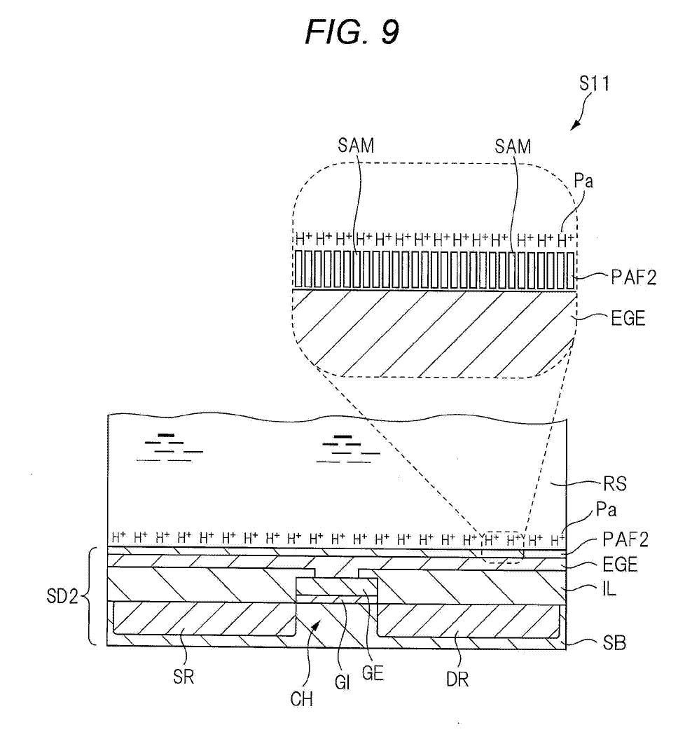

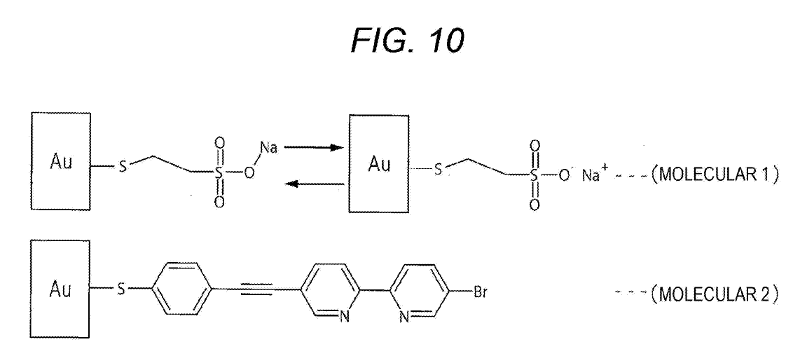

The configuration of the artificial olfactory sensing system of a second embodiment will be described using FIG. 9. FIG. 9 is an enlarged cross-sectional view illustrating main parts of the sensor unit S11 of the artificial olfactory sensing system of the second embodiment. FIG. 10 is a diagram schematically illustrating the molecules of a proton adsorption film PAF2 of the second embodiment.

In the artificial olfactory sensing system of the second embodiment, the configuration of the sensor unit which is one of the components is different from the configuration of the sensor unit of the artificial olfactory sensing system of the first embodiment. The other configurations of the artificial olfactory sensing system of the second embodiment are the same as those of the artificial olfactory sensing system of the first embodiment, and the redundant description will be omitted.

As illustrated in FIG. 9, the sensor unit S11 of the second embodiment includes a semiconductor device SD2, and the sensor cell C11 (not illustrated) which is disposed on the semiconductor device SD2. In the semiconductor device SD2 of the second embodiment, an extension gate electrode (first conductor film) EGE2 is formed on the gate electrode GE and on the insulating layer IL. Then, the proton adsorption film (first insulating film) PAF2 is formed on the extension gate electrode EGE2. The extension gate electrode EGE2 is made of a stacked film which forms a gold thin film (a film thickness of about 100 nm) on the aluminum film. Then, the proton adsorption film PAF2 is made of a self-assembled monolayer (SAM), and the proton Pa is adsorbed to the surface of the proton adsorption film PAF2.

As illustrated in FIG. 10, the self-assembled monolayer SAM is formed by Au—S bonding of the molecule of a thiol group and gold (Au) of the extension gate electrode EGE2. Examples of the molecules of the self-assembled monolayer SAM, there is a sodium 2-mercaptoethanesulfonate (MESA) (Molecule 1), or a Br-bipyridine-derivative (Molecule 2) of the thiol group. These molecules have a proton adsorption property. This configuration is a difference of the second embodiment from the first embodiment.

Further, in the sensor unit of the artificial olfactory sensing system of the second embodiment, the configurations of the sensor units other than the sensor unit S11 described above are similar to that of the sensor unit S11, and the redundant description will be omitted.

In addition, in the manufacturing method of the artificial olfactory sensing system of the second embodiment, the aluminum film (not illustrated) is formed on the gate electrode GE and on the insulating layer IL similar to the first embodiment, and patterned. Thereafter, the gold thin film is deposited on the patterned aluminum film to form the extension gate electrode EGE2. Subsequently, the surface of the extension gate electrode EGE2 is soaked one hour or more in 1 mM ethanol solution of the sodium 2-mercaptoethanesulfonate or the Br-bipyridine-derivative of the thiol group for example. With this configuration, the Au—S bonding is sequentially formed between the thiol group and Au. Therefore, the proton adsorption film PAF2 made of the self-assembled monolayer SAM can be formed on the extension gate electrode EGE2. The above point is a difference of the second embodiment from the first embodiment.

In the second embodiment, it can be seen that both the sodium 2-mercaptoethanesulfonate (Molecule 1) and the Br-bipyridine-derivative (Molecule 2) of the thiol group illustrated in FIG. 10 have the proton adsorption property. For example, as illustrated in FIG. 10, sodium ions are ionized in Molecule 1 to form sulfonic acid ions. Therefore, there is the proton adsorption property. In the second embodiment, the self-assembled monolayer SAM made of these molecules is employed as the proton adsorption film PAF2, so that the proton Pa can be adsorbed to the surface of the proton adsorption film PAF2 similar to the first embodiment.

Therefore, as described above, in a case where the positive ions in the physiological aqueous solution RS flow into the sensor cell C11, and the concentration of the positive ions in the physiological aqueous solution RS is lowered, the proton Pa adsorbed to the surface of the proton adsorption film PAF2 of the semiconductor device SD2 is dissociated from the proton adsorption film PAF2 and emitted into the physiological aqueous solution RS in order to compensate the positive ions in the physiological aqueous solution RS. As a result, the potential in the sensor cell C11 is shifted in the positive direction, and the potential of the surface SF of the proton adsorption film PAF1 is shifted in the negative direction. With this configuration, the potentials of the extension gate electrode EGE2 and the gate electrode GE integrally formed with the proton adsorption film PAF1 are shifted in the negative direction.

In this way, in the second embodiment, as the electrical response to the odor molecule, the potential change according to the change in amount of the protons Pa existing on the proton adsorption film PAF2 is measured similar to the first embodiment. As a result, as the electrical response to the odor molecule, the change in more stable potential can be observed with high sensitivity and reproducibility.

As described above, the proton adsorption film PAF2 of the second embodiment can be formed by soaking the surface of the extension gate electrode EGE2 into the ethanol solution. On the other hand, the proton adsorption film PAF1 of the first embodiment is formed by performing the oxygen plasma processing on the aluminum film. In other words, the entire substrate SB formed with the MOSFET is conveyed to a device which performs the oxygen plasma processing, and the whole substrate SB containing the MOSFET is exposed to oxygen plasma. Therefore, the second embodiment is advantageous compared to the first embodiment from the viewpoint of reducing the damage on the MOSFET caused by the oxygen plasma processing.

Further, in the second embodiment, the self-assembled monolayer SAM does not exist between the extension gate electrode EGE2 and the proton Pa adsorbed to the proton adsorption film PAF2. In other words, it is necessary for the self-assembled monolayer SAM to securely cover the extension gate electrode EGE2 in order to secure the insulation between the extension gate electrode EGE2 and the adsorbed proton Pa. If the insulation between the extension gate electrode EGE2 and the adsorbed proton Pa is not secured, the potential change according to the change in amount of the proton Pa is likely not to be measured correctly.

On the other hand, the proton adsorption film PAF1 of the first embodiment is an aluminum oxide film formed by the oxygen plasma processing. Since the aluminum oxide film operates as the insulating film, the insulation between the extension gate electrode EGE and the adsorbed proton Pa is sufficiently secured. In this viewpoint, the first embodiment is more advantageous than the second embodiment.

In addition, the proton adsorption film PAF1 of the first embodiment is a porous aluminum oxide film. On the other hand, the proton adsorption film PAF2 of the second embodiment is the self-assembled monolayer SAM formed in the surface of the gold thin film. Therefore, in the viewpoint of the proton adsorption amount, the first embodiment is more advantageous than the second embodiment.

Hitherto, the description has been given on the base of the embodiments according to the inventor. The invention is not limited to the embodiments, and various modifications can be made within a scope not departing from the spirit.

What is claimed is:

1. An artificial olfactory sensing system, comprising
a sensor unit which includes a transistor and a sensor cell, the sensor cell being configured such that an olfactory receptor is manifested on a lipid film,
wherein the transistor includes:
a substrate;
a source region and a drain region which are formed in the substrate; and
a gate electrode which is formed on the substrate between the source region and the drain region through a gate insulating film,
wherein a first insulating film is formed on the gate electrode,
wherein an electrolytic aqueous solution is disposed on the first insulating film,
wherein the sensor cell is disposed in the electrolytic aqueous solution,
wherein a proton is adsorbed on the first insulating film, and
wherein, when the olfactory receptor recognizes an odor molecule, positive ions in the electrolytic aqueous solution flow into the sensor cell from an ion channel provided in the olfactory receptor and, as a result, the proton is dissociated from the first insulating film into the electrolytic aqueous solution, and a potential of the gate electrode is changed.

2. The artificial olfactory sensing system according to claim 1,
wherein, on the gate electrode, a first conductor film is formed which is electrically connected to the gate electrode, and has an area larger than the sensor cell in top view,
wherein the first insulating film is formed in the same area as the first conductor film in top view, and
wherein the first insulating film comes into contact with the first conductor film.

3. The artificial olfactory sensing system according to claim 1,
wherein the first insulating film is made of an aluminum oxide film of which a surface is porous, and contains negative fixed charges.

4. The artificial olfactory sensing system according to claim 3,
wherein a hydroxyl group is bonded to an aluminum atom existing in the surface of the first insulating film.

5. The artificial olfactory sensing system according to claim 2,
wherein the first conductor film is a gold film, and
wherein the first insulating film is a self-assembled monolayer which is made of molecules bonded through Au—S bonding in the gold film.

6. The artificial olfactory sensing system according to claim 5,
wherein the molecule is made of a Br-bipyridine-derivative containing a thiol group or a sodium 2-mercaptoethanesulfonate.

7. The artificial olfactory sensing system according to claim 1,
wherein a stimulus time of the odor molecule is measured from a continuous time of a potential change of the gate electrode which is caused when the olfactory receptor recognizes the odor molecule.

8. The artificial olfactory sensing system according to claim 1,
wherein a potential change of the gate electrode which is caused when the olfactory receptor recognizes the odor molecule is differentiated with time to measure a concentration of the odor molecule.

9. The artificial olfactory sensing system according to claim 1,
wherein a plurality of the sensor units, a plurality of scanning lines, and a plurality of signal lines are included,
wherein the sensor unit is connected to one of the plurality of scanning and one of the plurality of signal lines,
wherein a plural types of the sensor cells exist, and
wherein the sensor unit containing the same types of sensor cells in the plurality of the sensor units is connected to the same scanning line among the plurality of scanning lines.

10. The artificial olfactory sensing system according to claim 9,
wherein the plurality of scanning lines are disposed to be crossed with the plurality of signal lines, respectively, and
wherein the plurality of the sensor units are disposed at intersections between the plurality of scanning lines and the plurality of signal lines.

11. A manufacturing method of an artificial olfactory sensing system, comprising:
(a) preparing a substrate in which a transistor is formed;
wherein the transistor includes
a source region and a drain region which are formed in the substrate, and
a gate electrode which is formed on the substrate between the source region and the drain region through a gate insulating film;
(b) forming a first conductor film on the gate electrode;
(c) forming a first insulating film on the first conductor film after the (b);
(d) disposing an electrolytic aqueous solution on the first insulating film after the (c); and
(e) disposing a sensor cell in which an olfactory receptor is manifested on a lipid film in the electrolytic aqueous solution after the (d), and forming a sensor unit,
wherein a proton is adsorbed on the first insulating film, and
wherein, when the olfactory receptor recognizes an odor molecule, positive ions in the electrolytic aqueous solution flow from an ion channel of the olfactory receptor to the sensor cell, and
wherein the proton is dissociated from the first insulating film into the electrolytic aqueous solution, and a potential of the gate electrode is changed.

12. The manufacturing method of an artificial olfactory sensing system according to claim 11,
wherein the first conductor film is made of an aluminum film,
wherein, in the (c),
oxygen plasma processing is performed on the aluminum film to form the first insulating film made of an aluminum oxide film.

13. The manufacturing method of an artificial olfactory sensing system according to claim 12,
wherein, in the (c),
the first insulating film is negatively charged by the oxygen plasma processing, and
wherein, in the (d),
the proton is adsorbed onto the first insulating film by disposing the electrolytic aqueous solution on the first insulating film.

14. The manufacturing method of an artificial olfactory sensing system according to claim 13,
wherein, in the (c),
the oxygen plasma process is performed on the aluminum film to form a chemical dangling bond in an aluminum atom existing in the surface of the aluminum film, and
wherein, in the (d),
the electrolytic aqueous solution is disposed on the first insulating film to cause a reaction between the aluminum atom of the chemical dangling bond and a water molecule in the electrolytic aqueous solution and, as a result, a hydroxyl group is bonded to the chemical dangling bond, and a proton generated by the reaction is hydrogen-bonded to the hydroxyl group.

15. The manufacturing method of an artificial olfactory sensing system according to claim 11,
wherein the first conductor film is made of gold film, and
wherein, in the (c),
the gold film is soaked to a solution containing molecules of a thiol group to form the first insulating film made of a self-assembled monolayer on the gold film.
